(19) United States
(12) Patent Application Publication
(10) Pub. No.: US 2023/0132922 A1
(43) Pub. Date: May 4, 2023

(54) NOVEL PROCESSES FOR THE PREPARATION OF RAPID MELT GRANULES

(71) Applicants: Chandanmal Pukhraj BOTHRA, Bangalore (IN); Hemanth Kumar BOTHRA, Bangalore (IN); Elayaraja NATARAJAN, Bangalore (IN); Siva Ram MUNNANGI, Bangalore (IN); Manmath Nana THALKARI, Bangalore (IN); Manivel THANGAPRAGASAM, Bangalore (IN); LYRUS LIFE SCIENCES PVT. LTD., Bangalore (IN); NOKHA TRADING LLP, Bangalore (IN)

(72) Inventors: Chandanmal Pukhraj Bothra, Bangalore (IN); Hemanth Kumar Bothra, Bangalore (IN); Elayaraja Natarajan, Bangalore (IN); Siva Ram Munnangi, Bangalore (IN); Manmath Nana Thalkari, Bangalore (IN); Manivel Thangapragasam, Bangalore (IN)

(21) Appl. No.: 17/911,948

(22) PCT Filed: Jul. 20, 2021
(86) PCT No.: PCT/IB2021/056541

§ 371 Date: Sep. 15, 2022

(30) Foreign Application Priority Data
Jul. 21, 2020 (IN) 202041031047

(51) Int. Cl. A61K 9/50; A61K 9/48; A61K 31/167; A61K 31/616; A61K 31/192; A61K 31/155; A61K 31/485; A61K 31/495; A61K 31/593; A61K 31/451; A61K 9/00; A61K 47/12; A61K 47/26; A61K 47/18; A61K 47/02

(52) U.S. Cl. CPC A61K 9/5089; A61K 9/5047; A61K 9/4866; A61K 31/167; A61K 31/616; A61K 31/192; A61K 31/155; A61K 31/485; A61K 31/495; A61K 31/593; A61K 31/451; A61K 9/0056; A61K 47/12; A61K 47/26; A61K 47/183; A61K 47/02; A61K 9/009

(57) ABSTRACT

The present invention relates to Rapid melt granules prepared by encapsulation and complexation processes. The present invention specifically relates to Rapid melt granules prepared by novel process of particulate coating comprising the steps of encapsulation of active ingredient with encapsulation polymer and/or pore former, drying, blending, optionally polymer coating, drying before blending and filling into sachets or compressing into tablets. The present invention more specifically relates to Rapid melt granules prepared by novel process of complexation comprising the steps of complexation of active ingredient with sulfonated polymers of ion exchange resin, drying, blending, optionally polymer coating, drying before blending and filling into sachets or compressing into tablets.

### FIELD OF INVENTION

The present invention relates to Rapid melt granules prepared by encapsulation and complexation processes.

The present invention specifically relates to Rapid melt granules prepared by novel process of particulate coating comprising the steps of encapsulation of active ingredient with encapsulation polymer and/or pore former, drying, blending, optional polymer coating, drying before blending and filling into sachets or compressing into tablets.

The present invention also relates to Rapid melt granules prepared by novel process of complexation comprising the steps of complexation of active ingredient with sulfonated polymers of ion exchange resin, optional encapsulation, drying, blending, optional polymer coating, drying before blending and filling into sachets or compressing into tablets.

### BACKGROUND OF INVENTION

It is well known that the most preferred dosage form for orally administration is a tablet or capsule. However, the requirement of a non-invasive delivery system persists due to patient's poor acceptance and compliance even with existing non-invasive conventional oral delivery systems.

Conventional oral drug delivery presents a drug with quick and full release that may go as such without producing the desired effect may be due to the presence of food, pH of the stomach, enzymatic degradation, change in GIT motility as so forth, giving not enough time to get absorbed. Patients with heart pumping issues and specialization population such as those undergoing dialysis or those who cannot consume more than certain amount of water find difficulty in administering conventional oral tablets. To overcome this, generally, tablets which disintegrate rapidly in the oral cavity (oral disintegrating tablets, orally disintegrating tablets) or films which dissolve rapidly in the oral cavity (intraoral strip formulations, oral strip technology) are used.

The oral administration route is considered to be the most widely used route because of its convenience of administration and manufacturing. Dosage forms, like orally disintegrating tablets (ODTs), mini tablets, and orally disintegrating mini tablets (ODMTs) are recognized as promising for use in pediatric patients. ODTs are known to be suitable drug delivery systems, especially for pediatric patients, because of their rapid disintegration properties, use without water, and no swallowing problems. However, ODT generally takes at least 30 seconds to completely disintegrate and dissolve in the oral cavity. In particular, ODT may be severely felt in the oral cavity until disintegration is completed.

There is need for developing pharmaceutical formulation in the form of quick-dissolving granules that dissolve quickly in the oral cavity and can be taken without water which does not give unpleasant feel in the mouth.

EP Publication No. 2 614 816 A1 discloses process for producing drug-containing granule comprising (a) a core particle that contains a drug causing bitterness, and (b) a masking coating that coats the core particle, wherein the masking coating contains: at least one polymer selected from methacrylic acid copolymer S, methacrylic acid copolymer L, methacrylic acid-ethyl acrylate copolymer, ethyl acrylate-methyl methacrylate copolymer, and ethyl acrylate-methyl methacrylate-ethyl ammonium trimethyl chloride methacrylate copolymer in 20 to 70 weight % of the coating; and at least one diluent selected from D-mannitol, lactose, trehalose, xylitol, maltitol, and erythritol in 40 to 250 weight % of the polymer.

US Publication No. 2005/0036977 A1 discloses method for preparing a taste-masked resinate oral dosage form comprising the steps of: forming a taste-masked resinate in an aqueous medium, wherein the resinate is a therapeutically effective amount of a water-insoluble, bitter-tasting pharmaceutically active substance complexed with an ion-exchange resin, wherein the aqueous medium comprises water and, optionally, one or more of the following: neutralizing agents, sweetening agents, flavoring agents, coloring agents, anti-foaming agents and the like or mixtures thereof, wherein the water-insoluble active substance is present in a water-insoluble base form and is complexed with the resin in a taste-masking effective amount, wherein the ion-exchange resin is a cationic ion-exchange resin.

US Publication No. 2006/0127479 A1 discloses process for preparing a taste masked pharmaceutical composition, said process comprising: (i) preparing an aqueous solution or dispersion which comprises a bitter tasting drug and a binder; (ii) applying the solution or dispersion formed in Step (i) onto an inert carrier to form core granules or agglomerates; (iii) preparing a coating comprising an aqueous dispersion of a pharmaceutically acceptable cationic co-polymer based on mono- or dialkylaminoalkyl methacrylate and neutral acrylic or methacrylic esters, wherein the alkyl group independently has 1 to 6 carbon atoms; and (iv) applying the coating formed in Step (iii) to the surface of the core granules or agglomerates formed in Step (ii) to form a composition, wherein the process is essentially free of an organic solvent.

US Publication No. 2006/0182802 A1 discloses method for the orally disintegrable tablet having the fine granules of the coated cores comprises: (i) coating a core comprising crystalline cellulose and lactose with a physiologically active substance and an excipient, followed by being coated with a coating layer comprising a water-soluble polymer to obtain a composition, (ii) coating the resultant composition with an enteric coating layer having polyethyleneglycol, and then with an enteric coating layer having triethyl citrate, and then followed by being coated by mannitol to obtain fine granule, and (iii) blending the resultant fine granule with an additive, followed by molding.

US Publication No. 2010/0136110 A1 discloses process of manufacturing the granular pharmaceutical composition for oral administration comprising the steps of: (1) forming a layer comprising a water-soluble insolubilizer and a water-soluble insolubilizing substance outside a drug-containing particle, and (2) coating the obtained particle with (i) a coating consisting of a methyl methacrylate-butyl methacrylate-dimethylaminoethyl methacrylate copolymer and a water-soluble polymer, or (ii) a coating consisting of a methyl methacrylate-butyl methacrylate-dimethylaminoethyl methacrylate copolymer, a water-soluble polymer and a glidant.

US Publication No. 2012/0076858 A1 discloses method of preparing an orally disintegrating dosage form, the method comprising: preparing an aqueous polymer dispersion, and coating the dispersion onto an active ingredient, wherein the dispersion is obtained by free-radical emulsion polymerization of a monomer mixture comprising N,N-diethylaminoethyl methacrylate, and at least one compound selected from esters of α,β-ethlenically unsaturated mono- and dicarboxylic acids with C₁-C₈ alkanols.

US Publication No. 2016/0296463 A1 discloses process of preparing drug/polymer solid solution/dispersion wherein salt form of the drug is neutralized in whole or in-part, to form a neutral or free base form of the drug prior to removal of the solvent from the polymer solution and drug mixture, the ratio of polymer to drug should be at a level that prevents precipitation of the drug from the polymer solution and drug mixture during neutralization with an acidifying or alkalizing composition or solution.

The inventors of present invention provide Rapid melt granules prepared by novel process using particulate coating comprising the steps of encapsulation of active ingredient with encapsulation polymer and/or pore former, drying, blending, optional polymer coating, drying before blending and filling into sachets. The inventors of present invention also provide Rapid melt granules prepared by novel process using complexation comprising the steps of complexation of active ingredient with sulfonated polymers of ion exchange resin, optional encapsulation, drying, blending, optional polymer coating, drying before blending and filling into sachets or compressing into tablets.

The invention aids in easy administration and no suffocation risk resulting from physical obstruction by a solid dosage form especially to more general patients requiring daily medication. Provides a smooth mouth feel while providing an excellent taste barrier. Rapidly dispersed within seconds (10 to 15 seconds) in the oral cavity. No water required for administration. Can be easily taken on the go. Paediatrics and geriatric populations are the primary targets as both the groups find it difficult to swallow conventional tablets. This is suitable for wide range of product including acute and chronic pain management, stroke management, cough suppression, pulmonary arterial hypertension, anti-diarrhoeal, anti-emetics, anti-histamine etc. This technology is beneficial for critical care patients and also for emergency use such as anti-diarrhoeal management, anti-emetics during travelling which can be easily taken without water.

### OBJECTIVE OF INVENTION

The main objective of the present invention is to provide taste masked Rapid melt granules prepared by encapsulation and complexation processes.

Another objective of the present invention is to provide Rapid melt granules which dissolves in the oral cavity within 10 to 15 seconds.

Another objective of the present invention is to provide Rapid melt granules prepared by novel process of particulate coating comprising the steps of encapsulation of active ingredient with encapsulation polymer and/or pore former, drying, blending, optional polymer coating, drying before blending and filling into sachets or compressing into tablets.

Another objective of the present invention is to provide Rapid melt granules prepared by novel process of complexation comprising the steps of complexation of active ingredient with sulfonated polymers of ion exchange resin, optional encapsulation, drying, blending, optional polymer coating, drying before blending and filling into sachets or compressing into tablets.

Yet another objective of the present invention is to provide Rapid melt granules which melt in the mouth without providing bitter mouth feel and provide a faster onset of action (including buccal, pharyngeal absorption).

### SUMMARY OF INVENTION

Accordingly, the present invention provides processes for preparing Rapid melt granules which can mask taste of unpleasant active ingredients.

One embodiment of the present invention provides processes for preparing Rapid melt granules which disperse within 20 seconds in the oral cavity.

Another embodiment of the present invention provides processes for preparing Rapid melt granules which melts in the mouth to provide a faster onset of action (including buccal, pharyngeal absorption) and avoiding any first pass metabolism.

Another embodiment of the present invention provides processes for preparing Rapid melt granules which can be taken without water.

Another embodiment of the present invention provides processes for preparing Rapid melt granules having no foreign body, no residual feeling and which gives refreshing feeling.

Another embodiment of the present invention provides Rapid melt granules in the oral cavity prepared by encapsulation and complexation processes.

Another embodiment of the present invention provides Rapid melt granules prepared by novel process of particulate coating comprising the steps of encapsulation of active ingredient with encapsulation polymer and/or pore former, drying, blending, optionally polymer coating, drying before blending and filling into sachets or compressing into tablets.

Another embodiment of the present invention provides a novel process for the preparation of rapid melt granules using particulate coating comprising the steps of encapsulation of active ingredient with encapsulation polymer and/or pore former, drying, blending with one or more excipients selected from diluents, glidants, salivating agent, flavours, natural or artificial sweeteners.

Another embodiment of the present invention provides Rapid melt granules prepared by novel process of complexation comprising the steps of complexation of active ingredient with sulfonated polymers of ion exchange resin, optional encapsulation, drying, blending, optional polymer coating, drying before blending and filling into sachets or compressing into tablets.

Another embodiment, the present invention provides a process for preparing Rapid melt granules using particulate coating, wherein said process comprising steps of:
(a) coating of active ingredient using low viscous swellable or non swellable polymers and/or pore former and optionally other pharmaceutically acceptable excipients in a suitable equipment,
(b) drying and sifting of the coated granules obtained in step (a),
(c) blending of coated granules with excipients such as diluents, glidants, flavours, natural or artificial sweeteners, salivating agent, buffering agents etc, and
(d) filling the obtained blend of granules into sachets or compressing into tablets.

Another embodiment, the present invention provides a process for preparing Rapid melt granules, wherein said process comprising steps of:
(a) coating of active ingredient using ethyl cellulose, Isopropyl alcohol and water solution in a fluid bed processor,
(b) drying and sifting of the coated granules obtained in step (a),
(c) blending of coated granules with excipients such as maltodextrose, mannitol, xylitol, citric acid monohydrate, Aspartame and lime flavour,
(d) filling the obtained blend of granules into sachets or compressing into tablets.

Another embodiment, the present invention provides a process for preparing Rapid melt granules using particulate coating, wherein said process comprising steps of:
(a) coating of active ingredient using low viscous swellable or non swellable polymers and/or pore former and optionally other pharmaceutically acceptable excipients in a suitable equipment,
(b) drying and sifting of the coated granules obtained in step (a),
(c) coating of dried granules if required using a polymer solution containing plasticizers, anti-tacking agents and optionally buffering agents, flavours and natural or artificial sweeteners,
(d) optionally drying and sifting of the coated granules obtained in step (b) or step (c),
(e) blending of coated granules with excipients such as diluents, glidants, flavours, natural or artificial sweeteners, buffering agents etc, and
(f) filling the obtained blend of granules into sachets or compressing into tablets.

Another embodiment, the present invention provides a process for preparing Rapid melt granules, wherein said process comprising steps of:
(a) dissolving hydroxypropyl methylcellulose in purified water,
(b) coating of active ingredient and Mannitol mixture using hydroxypropyl methylcellulose solution,
(c) coating the hydroxypropyl methylcellulose coated granules with ethyl cellulose, Isopropyl alcohol and water solution in a fluid bed processor,
(d) drying and sifting of the coated granules obtained in step (c),
(e) blending of coated granules with talc, maltodextrin, xylitol, aspartame and lime flavour,
(f) filling the obtained blend of granules into sachets or compressing into tablets.

Another embodiment, the present invention provides a process for preparing Rapid melt granules, wherein said process comprising steps of:
(a) complexing of active ingredient with sulfonated polymers of ion exchange resin,
(b) draining the supernatant solution and collecting the residue,
(c) drying and sifting of complexed granules,
(d) blending of complexed granules with excipients such as diluents, glidants, flavours, natural or artificial sweeteners, buffering agents etc, and
(e) filling the obtained blend of complexed granules into sachets or compressing into tablets.

Another embodiment, the present invention provides a process for preparing Rapid melt granules, wherein said process comprising steps of:
(a) complexing of active ingredient with sodium polystyrene sulfonate,
(b) draining the supernatant solution and collecting the residue,
(c) drying and sifting of complexed granules,
(d) blending of complexed granules with talc, Aspartame, Sorbitol, Peach flavour, and Maltodextrin, and
(e) filling the obtained blend of complexed granules into sachets or compressing into tablets.

Another embodiment, the present invention provides a process for preparing Rapid melt granules, wherein said process comprising steps of:
(a) complexing of active ingredient with sulfonated polymers of ion exchange resin,
(b) draining the supernatant solution and collecting the residue,
(c) drying and sifting of complexed granules,
(d) coating the complex with using low viscous swellable or non swellable polymers and/or pore former,
(e) blending of complexed granules with excipients such as diluents, glidants, flavours, natural or artificial sweeteners, buffering agents etc, and
(f) filling the obtained blend of complexed granules into sachets or compressing into tablets.

Another embodiment, the present invention provides a process for preparing Rapid melt granules, wherein said process comprising steps of:
(a) complexing of active ingredient with sodium polystyrene sulfonate,
(b) draining the supernatant solution and collecting the residue,
(c) drying and sifting of complexed granules with ethyl cellulose with suitable solvent,
(d) coating the complexed granules with ethyl cellulose, isopropyl alcohol and purified water solution,
(e) blending of coated complexed granules with aspartame, xylitol, orange flavour, mannitol and talc, and
(f) filling the obtained blend of complexed granules into sachets or compressing into tablets.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The oral route of drug administration is the most common route for systemic effect of drug. Solid dosage forms are most popular because ease of administration, accurate dosage, self-medication, pain avoidance and most importantly patient compliance. The most commonly used solid dosage forms are tablets and capsules; one of the drawback for some patients, difficulty to swallow. Drinking water plays important role for swallowing of oral dosage forms. Often times people experience inconvenience in swallowing conventional dosage forms such as water whereas water is not available in case of motion sickness (kenetosis) and sudden episodes of coughing during common cough and cold, allergic condition and bronchitis.

Rapid-release tablets, also called rapidly dissolving tablets, rapidly disintegrating tablets, orally-dispersible tablets, quick disintegrating tablets, mouth dissolving tablets, fast disintegrating tablets, fast-dissolving tablets, rapid-dissolving tablets, or porous tablets are characterized by disintegrating or dissolving in the mouth within 1 minute.

Taste-masking techniques are applied to mask or overcome the bitter or unpleasant taste of active pharmaceutical ingredients/drugs to achieve patient acceptability and compliance. Oral administration of bitter or unpleasant tasting drugs is often the biggest barrier for patient groups, such as pediatrics and geriatrics.

The commonly used industrial techniques/methods of taste-masking include organoleptic methods, polymer coating, hot-melt extrusion, microencapsulation, complexation, and spray-drying.

The present invention provides processes for preparing taste masked Rapid melt granules which dissolve in oral cavity within 20 seconds.

This technology is suitable for wide range of product including acute and chronic pain management, stroke management, cough suppression, pulmonary arterial hypertension, anti-diarrhoeal, anti-emetics, anti-histamine etc. This technology is beneficial for critical care patients and also for emergency use such as anti-diarrhoeal management, anti-emetics during travelling which can be easily taken without water.

The term “active ingredient” of the present invention includes ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Preferably used active ingredient includes anti-inflammatory, antipyreutic, analgesic, antithrombotic, antidiarrheal agent, antihistamine and antidiabetic agents.

Most preferably used active ingredient includes Aspirin, Loperamide Hydrochloride, Cetirizine Hydrochloride, Fexofenadine hydrochloride, Vitamin-C, Vitamin-D, D-mannose, Curcumin with and without zinc, Galantamine Hydrobromide, Tramadol Hydrochloride, Methylphenidate hydrochloride, Ranitidine Hydrochloride, Paracetamol, Ibuprofen and its salts, Metformin Hydrochloride, Lornoxicam, Piroxicam, Diphenhydramine hydrochloride, Guaifenesin, Bromhexine Hydrochloride, Ondansetron hydrochloride, Sildenafil Citrate, Tadalafil, Phenylephrine, Prochlorperazine maleate, Indomethacin, Diclofenac, Celecoxib, Trazodone, Procyclidine hydrochloride, Melatonin, Oxybutynin hydrochloride and Dextromethorphan Hydrobromide.

The concentration of the active ingredient used in the compositions of the present invention is in the range of 0.1% to 55% (w/w). Preferably used concentration of active ingredient is from 0.2% to 50% (w/w).

Encapsulating polymers or polymers or pore former used alone or in combination in the compositions of the present invention include, but are not limited to hydroxypropyl methylcellulose, Ethyl cellulose, PEG, Eudragits, Polyvinyl alcohols, Polyvinyl acetate, hydroxypropylcellulose, hydroxypropyl-methylcellulose phthalate, cellulose acetate phthalate, starch etc. Preferably used polymers include starch, ethyl cellulose and hydroxypropyl methylcellulose.

The concentration of encapsulating polymers or polymers or pore former used in the compositions of the present invention is in the range of 0.1% to 30% (w/w). Preferably used concentration of encapsulating polymers or polymers or pore former is 0.5% to 25% (w/w).

Sulfonated polymers of ion exchange resin used in the compositions of the present invention include, but are not limited to weakly acidic Amberlite® IRP-64 (Polacrilex Resin), strongly acidic Amberlite® IRP-69 (Sodium Polystyrene Sulfonate), weakly acidic Amberlite® IRP-88 (Polyacrylic Potassium), Purolite®, strongly basic Duolite1 M AP-143 (Cholestyramine Resin USP), strongly acidic Dowex® 50WX series and strongly basic Dowex® IX series. Preferably used sulfonated polymers of ion exchange resin include Polacrilex Resin (Amberlite® IRP-64) and Sodium Polystyrene Sulfonate (Amberlite® IRP-69). Most preferably Amberlite® IRP-69 (Rohm and Haas), which is sulfonated polymers composed of polystyrene crosslinked with 8% of divinylbenzene.

The concentration of sulfonated polymers of ion exchange resin used in the compositions of the present invention is in the range of 1% to 50% (w/w). Preferably used concentration of sulfonated polymers of ion exchange resin is 2% to 40% (w/w).

The term “pharmaceutically acceptable excipients” include diluents, glidants, flavours, natural or artificial sweeteners, buffering agents etc.

Diluents used alone or in combination in the compositions of the present invention include, but are not limited to silicon dioxide, sugars, starches, lactose, sucrose, sorbitol, fructose, talc, stearic acid, magnesium aluminium metasilicate, dicalcium phosphate, erythitol, xylitol, mannitol, maltitol, isomalt, dextrose, maltose, lactose, microcrystalline celluloses and mixtures thereof. Preferably used diluents include Mannitol, Xylitol, Sorbitol and Maltodextrin.

Diluent as used herein in the compositions of the present invention is in the range of 1% to 95% (w/w). Preferably used concentration of diluent is from 5% to 90% (w/w).

Glidants used alone or in combination in the compositions of the present invention include, but are not limited to talc, magnesium silicate, colloidal silicon dioxide, amorphous silicon dioxide and calcium silicate. Preferably used glidant is talc.

The concentration of glidant used in the compositions of the present invention is in the range of 0.1% to 3% (w/w). Preferably used concentration of glidant in the compositions of the present invention is from 0.5% to 2% (w/w).

Plasticizers used alone or in combination in the compositions of the present invention include, but are not limited to benzyl benzoate, chlorobutanol, dibutyl sebacate, diethyl phthalate, glycerol, polyethylene glycol, sorbitol, triacetin and triethyl citrate.

The concentration of plasticizer used in the compositions of the present invention is in the range of 0.1% to 20% (w/w). Preferably used concentration of plasticizer used in combination in the compositions of the present invention is from 0.5% to 10% (w/w).

Anti-tacking agents used alone or in combination in the compositions of the present invention include, but are not limited to talc, magnesium stearate, calcium docosanoate, stearic acid, calcium arachinate, hydrogenated castor oil, or triglycerides.

The concentration of anti-tacking agents used in the compositions of the present invention is in the range of 0.1% to 20% (w/w). Preferably used concentration of anti-tacking agents used in combination in the compositions of the present invention is from 0.5% to 10% (w/w).

Buffering agents used alone or in combination in the compositions of the present invention include, but are not limited to sodium carbonate, sodium bicarbonate, potassium carbonate, potassium bicarbonate, sodium phosphate dibasic, sodium phosphate tribasic, potassium phosphate dibasic, potassium phosphate tribasic, calcium carbonate, magnesium carbonate, sodium hydroxide, magnesium hydroxide, potassium hydroxide, aluminium hydroxide, and combinations thereof.

Flavours used alone or in combination in the compositions of the present invention include, but are not limited to volatile oils, synthetic flavour oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors. Preferably used flavours include cherry flavor, Lime flavor and Peach flavor.

The concentration of flavour used in the compositions of the present invention is in the range of 0.01% to 2% (w/w). Preferably used concentration of favouring agent in the compositions of the present invention is from 0.1% to 0.5% (w/w).

Sweeteners used alone or in combination in the compositions of the present invention include, but are not limited to fructose (corn syrup), dextrose, invert sugar, fructose, and mixtures thereof; acesulfame potassium, saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, xylitol, and the like.

The concentration of sweeteners used in the compositions of the present invention is in the range of 0.1% to 5% (w/w). Preferably used concentration of sweetener in the compositions of the present invention is from 0.1% to 3% (w/w).

Salivating agent used alone or in combination in the compositions of the present invention include, but are not limited to citric acid, malic acid, tartaric acid, food salts such as sodium chloride and salt substitutes, potassium chloride, and mixtures thereof. Preferably used salivating agent is citric acid monohydrate.

The concentration of salivating agent used in the compositions of the present invention is in the range of 0.1% to 5% (w/w). Preferably used concentration of salivating agent in the compositions of the present invention is from 0.1% to 3% (w/w).

Rapid melt granules preparation of present invention has been prepared by using particulate coating and complexation techniques.

The present invention is illustrated in detail but not limiting to, the following examples. It will be apparent to those skilled in the art that many modifications, both to materials and methods, may be practiced without departing from the scope of the invention.

### Example 1: Encapsulation Using Polymer: Particulate Coating

| S. No | Ingredients | Quantity (% w/w) |
|---|---|---|
| 1. | Aspirin | 8.10 |
| 2. | Ethyl cellulose | 0.50 |
| 3. | Citric acid monohydrate | 3.50 |
| 4. | Maltodextrose | 35.30 |
| 5. | Mannitol | 26.25 |
| 6. | Xylitol | 26.25 |
| 7. | Aspartame | 0.50 |
| 8. | Lime flavor | 0.10 |
| 9. | Purified water | QS |
| 10. | Isopropyl alcohol | QS |

Manufacturing Process:

Ethyl cellulose is dissolved in IPA:Water (90:10 ratio) under continuous stirring. Aspirin was loaded into a fluid bed processor and coated with ethyl cellulose solution to attain fine coated granules. The coated granules along with maltodextrose, mannitol, xylitol and citric acid monohydrate were mixed in suitable blender for 20 minutes. Aspartame and lime flavour were added and blended for 5 minutes. Obtained granules were packaged into appropriate containers/sachets or compressed into tablets.

Stability testing: The Aspirin mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 1 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |

Related Substances (%)

| | | | |
|---|---|---|---|
| Unknown impurity | 0.060 | 0.120 | 0.102 |
| Impurity - A | 0.150 | 0.172 | 0.168 |
| Total impurities | 0.180 | 0.310 | 0.291 |
| Assay (%) | 99.6 | 98.9 | 100.8 |
| Water content | 3.6% | 4.5% | 3.9% |

### Example 2: Encapsulation Using Polymer: Particulate Coating

| S. No | Ingredients | Quantity (% w/w) |
|---|---|---|
| 1. | Paracetamol | 31.25 |
| 2. | Mannitol | 31.25 |
| 3. | Hydroxypropyl methylcellulose HPMC | 7.50 |
| 4. | Ethyl cellulose | 10.00 |
| 5. | Aspartame | 1.00 |
| 6. | Xylitol | 15.00 |
| 7. | Cherry flavor | 0.25 |
| 8. | Maltodextrin | 12.50 |
| 9. | Talc | 1.25 |
| 10. | Purified water | QS |
| 11. | Isopropyl alcohol | QS |

Manufacturing Process:

HPMC is dissolved in sufficient amount of purified water under continuous stirring. Paracetamol and Mannitol were loaded into fluid bed processor and coated using HPMC solution to attain fine coated granules. Ethyl cellulose is dissolved in IPA:Water (90:10 ratio) under continuous stirring. HPMC coated granules were coated with ethyl cellulose solution in fluid bed processor. Ethyl cellulose coated granules were blended with talc, maltodextrin, xylitol, aspartame and cherry flavour in a blender for 15 minutes. Obtained granules were packaged into appropriate containers/sachets or compressed into tablets.

Stability testing: The paracetamol mouth dissolving compositions manufactured and stability studied for 6-month at the conditions 25° C./60% relative humidity and 40° C./75% relative humidity, respectively.

Stability Results:

Pack: Alu- Alu pouch

Stability data of Example - 2

| | | |
|---|---|---|
| | 40° C./75% | 25° C./60% |
| Initial | (6 months) | (6 months) |

Description

| | | | |
|---|---|---|---|
| | White | White | White |
| | granular | granular | granular |
| Parameters | powder | powder | powder |

Related substance

| | | | |
|---|---|---|---|
| Imp - J | ND | ND | ND |
| (4- Chloroacetanilide) | | | |
| Imp - K | ND | ND | ND |
| (4-Aminophenol) | | | |
| Any other impurities | 0.001 | 0.004 | 0.003 |
| Total impurities | 0.004 | 0.010 | 0.007 |
| Assay (%) | 101.1 | 100.6 | 100.2 |
| Water contents | 4.6% | 4.9% | 4.3% |

### Example 3: Encapsulation Using Polymer: Particulate Coating

| S. No | Ingredients | Quantity (% w/w) |
|---|---|---|
| 1. | Ibuprofen sodium | 50.00 |
| 2. | Lactose | 25.00 |
| 3. | Starch | 10.00 |
| 4. | Ethyl cellulose | 15.00 |
| 5. | Aspartame | 1.50 |
| 6. | Xylitol | 20.00 |
| 7. | Lime flavor | 0.30 |
| 8. | Maltodextrin | 13.00 |
| 9. | Talc | 1.00 |
| 10. | Purified water | QS |
| 11. | Isopropyl alcohol | QS |

Manufacturing Process:

Starch is dissolved in sufficient amount of purified water under continuous stirring. Ibuprofen sodium and Lactose were loaded into fluid bed processor and coated using starch solution to attain fine coated granules. Ethyl cellulose was dissolved in IPA:water (90:10 ratio) under continuous stirring. Starch coated granules were coated with obtained ethyl cellulose solution. Granules were dried. Ethyl cellulose coated granules were blended with talc, maltodextrin, xylitol, aspartame and lime flavour in a blender for 15 minutes. Obtained granules were packaged into appropriate containers/sachets or compressed into tablets.

Stability testing: The ibuprofen mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 3 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |

Related substance

| | | | |
|---|---|---|---|
| 4-Isobutyl acetophenon | ND | ND | ND |
| Impurity J | 0.050 | 0.058 | 0.051 |
| Max. Unspecified | 0.047 | 0.047 | 0.047 |
| Total impurities | 0.109 | 0.116 | 0.111 |
| Assay (%) | 100.5 | 99.6 | 102.3 |
| Water contents | 3.6% | 3.9% | 3.3% |

### Example 4: Encapsulation Using Polymer: Particulate Coating

| S. No | Ingredients | Quantity (% w/w) |
|---|---|---|
| 1. | Ibuprofen sodium | 50.00 |
| 2. | Lactose | 5.00 |
| 3. | Starch | 5.00 |
| 4. | Ethyl cellulose | 20.00 |
| 5. | Aspartame | 1.50 |
| 6. | Xylitol | 20.00 |
| 7. | Lime flavor | 0.30 |
| 8. | Maltodextrin | 13.00 |
| 9. | Talc | 1.00 |
| 10. | Purified water | QS |
| 11. | Isopropyl alcohol | QS |

Manufacturing Process:

Starch and lactose is dissolved in sufficient amount of purified water under continuous stirring. Ibuprofen sodium was loaded into fluid bed processor and coated using starch and lactose dispersion to attain fine coated granules. Ethyl cellulose was dissolved in IPA:water (90:10 ratio) under continuous stirring. Starch and lactose coated granules were coated with obtained ethyl cellulose solution. Granules were dried. Ethyl cellulose coated granules were blended with talc, maltodextrin, xylitol, aspartame and lime flavour in a blender for 15 minutes. Obtained granules were packaged into appropriate containers/sachets or compressed into tablets.

Stability testing: The ibuprofen mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 4 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |

Related substance (%)

| | | | |
|---|---|---|---|
| 4-Isobutyl acetophenon | ND | ND | ND |
| Impurity J | 0.053 | 0.059 | 0.055 |
| Max. Unspecified | 0.043 | 0.043 | 0.043 |
| Total impurities | 0.105 | 0.120 | 0.112 |
| Assay (%) | 100.5 | 99.6 | 102.3 |
| Water content | 3.2% | 3.1% | 3.0% |

### Example 5: Encapsulation Using Ion Exchange Resin: Complexation Process

| S. No | Ingredients | Quantity (% w/w) |
|---|---|---|
| 1. | Metformin HCl | 38.2 |
| 2. | Sodium Polystyrene Sulfonate | 38.2 |
| | (Amberlite ® IRP-64) | |
| 3. | Aspartame | 1.5 |
| 4. | Sorbitol | 13.4 |
| 5. | Peach flavor | 0.2 |
| 6. | Maltodextrin | 7.6 |
| 7. | Talc | 0.9 |
| 8. | Purified water | QS |

Manufacturing Process:

Metformin Hydrochloride was dissolved in purified water under continuous stirring. Amberlite® IRP-64 was dispersed in the obtained above solution and stirred continuously for 5 hours. Dispersion was kept for standing for few hours and the supernatant solution was decanted. Drug-resin complex residue was collected and dried in a tray drier. Drug-resin complex was blended with talc, Aspartame, Sorbitol, Peach flavour, Maltodextrin in a blender for 15 minutes. Obtained granules are packaged into appropriate containers/sachets or compressed into tablets.

Stability testing: The metformin mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 5 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |

Related substance

| | | | |
|---|---|---|---|
| 1- Cyanoguanidine | 0.0043% | 0.0048% | 0.0046% |
| Any Unknown Impurity | 0.0096% | 0.0096% | 0.0095% |
| Total Impurities | 0.0476% | 0.0586% | 0.0560% |
| Assay (%) | 100.1 | 100.6 | 100.9 |
| Water contents | 4.2% | 5.1% | 5.0% |

### Example 6: Encapsulation Using Complexing Ion Exchange Resin and Complexing Polymer: Complexation Process

| S. No | Ingredients | Quantity (% w/w) |
|---|---|---|
| 1. | Dextromethorphan HBr | 7.89 |
| 2. | Sodium Polystyrene Sulfonate | 7.89 |
| | (Amberlite ® IRP-69) | |
| 3. | Ethyl cellulose | 2.63 |
| 4. | Aspartame | 1.32 |
| 5. | Xylitol | 52.63 |
| 6. | Orange flavour | 0.26 |
| 7. | Mannitol | 26.32 |
| 8. | Talc | 1.05 |
| 9. | Purified water | QS |
| 10. | Isopropyl alcohol | QS |

Manufacturing Process:

Dextromethorphan Hydrobromide was dissolved in purified water under continuous stirring. Amberlite® IRP-64 was dispersed into the above solution and stirred continuously for 5 hours. Dispersion was kept for standing for few hours and the supernatant solution was decanted. Drug-resin complex residue was collected and dried in a tray drier. Ethyl cellulose was dissolved in IPA:Water (90:10 ratio) under continuous stirring. Drug-resin complex was loaded into a fluid bed processor and coated with ethyl cellulose solution. Drug-resin complex granules were dried. Drug-resin complex granules were blended with Aspartame, Xylitol, Orange flavour, Mannitol, Talc in a blender for 15 minutes. Obtained granules were packaged into appropriate containers/sachets or compressed into tablets.

Stability testing: The Dextromethorphan mouth dissolving compositions manufactured and stability studied for 6-month at the conditions 25° C./60% relative humidity and 40° C./75% relative humidity, respectively.

Stability Results:

Pack: Alu- Alu pouch

Stability data of Example - 6

| | | |
|---|---|---|
| | 40° C./75% | 25° C./60% |
| Initial | (6 months) | (6 months) |

Description

| | | | |
|---|---|---|---|
| | White | White | White |
| | granular | granular | granular |
| Parameters | powder | powder | powder |

Related substance (%)

| | | | |
|---|---|---|---|
| Imp - C | 0.063 | 0.069 | 0.062 |
| Unspecified impurity | 0.046 | 0.070 | 0.058 |
| Total Impurities | 0.157 | 0.221 | 0.199 |
| Assay (%) | 100.9 | 99.6 | 98.0 |
| Water contents | 4.2% | 4.8% | 5.1% |

### Example 7: Encapsulation Using Complexing Ion Exchange Resin and Complexing Polymer: Complexation Process

| S. No | Ingredients | Quantity (% w/w) |
|---|---|---|
| 1. | Cetirizine hydrochloride | 2.00 |
| 2. | Sodium Polystyrene Sulfonate | 2.00 |
| | (Amberlite ® IRP-69) | |
| 3. | Ethyl cellulose | 0.50 |
| 4. | Sucralose | 1.00 |
| 5. | Maltodextrin | 10.00 |
| 6. | Lemon flavour | 0.26 |
| 7. | Mannitol | 10.00 |
| 8. | Talc | 1.00 |
| 9. | Purified water | QS |
| 10. | Isopropyl alcohol | QS |

Manufacturing Process:

Cetirizine hydrochloride was dissolved in purified water under continuous stirring. Amberlite® IRP-64 was dispersed into the above solution and stirred continuously for 5 hours. Dispersion was kept for standing for few hours and the supernatant solution was decanted. Drug-resin complex residue was collected and dried in a tray drier. Ethyl cellulose was dissolved in IPA:water (90:10 ratio) under continuous stirring. Drug-resin complex was loaded into fluid bed processor and coated with ethyl cellulose solution. Drug-resin complex granules were dried. Drug-resin complex granules were blended with Sucralose, Maltodextrin, Lemon flavor, Mannitol, Talc in a blender for 15 minutes. Obtained granules are packaged into appropriate containers/sachets or compressed into tablets.

Stability testing: The cetirizine mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 7 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |

Related substance

| | | | |
|---|---|---|---|
| Impurity A | 0.004% | 0.008% | 0.006% |
| Impurity B | Not detected | Not detected | Not detected |
| Impurity G | 0.06% | 0.12% | 0.06% |
| Any unspecified | Below | Below | Below |
| impurity | disregard limit | disregard limit | disregard limit |
| Total impurities | 0.06% | 0.12% | 0.06% |
| Assay (%) | 99.8 | 100.2 | 100.6 |
| Water contents | 4.8% | 5.2% | 4.9% |

### Example 8: Encapsulation Using Polymer: Particulate Coating

| S. No | Ingredients | Quantity (% w/w) |
|---|---|---|
| 1. | Vitamin - D | 15.225 |
| 2. | Mannitol | 15.000 |
| 3. | Ethyl cellulose | 5.000 |
| 4. | Mannitol SD 100 | 30.775 |
| 5. | Aspartame | 3.000 |
| 6. | Xylisorb 300 | 30.000 |
| 7. | Pine apple flavour | 1.000 |
| 8. | Purified water | QS |
| 9. | Isopropyl alcohol | QS |

Manufacturing Process:

Vitamin-D is dissolved in sufficient amount of isopropyl alcohol under continuous stirring. Mannitol were loaded into fluid bed processor, sprayed Vitamin-D solution and coated using ethyl cellulose solution to attain fine coated granules. Ethyl cellulose was dissolved in IPA:water (90:10 ratio) under continuous stirring. Granules were dried. Ethyl cellulose coated granules were blended with Mannitol SD 100, Aspartame Xylisorb 300, Pine apple flavour in a blender for 15 minutes. Obtained granules were packaged into appropriate containers/sachets or compressed into tablets.

Stability testing: The Vitamin-D mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 8 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |
| Assay (%) | 99.6 | 90.2 | 97.2 |
| Water contents | 3.8% | 4.2% | 4.1% |

### Example 9: Encapsulation Using Polymer: Particulate Coating

| S. No | Ingredients | Quantity (% w/w) |
|---|---|---|
| 1. | Sodium ascorbate | 7.28 |
| 2. | Ascorbic acid | 1.42 |
| 3. | Ethyl cellulose | 1.00 |
| 4. | Maltodextrin | 9.89 |
| 5. | Mannitol 160C | 55.10 |
| 6. | Aspartame | 1.97 |
| 7. | Aerosil | 0.10 |
| 8. | Xylitol | 23.0 |
| 9. | Orange flavour | 0.24 |
| 10. | Purified water | QS |
| 11. | Isopropyl alcohol | QS |

Manufacturing Process:

Sodium ascorbate and Ascorbic acid were loaded into fluid bed processor and coated using ethyl cellulose solution to attain fine coated granules. Ethyl cellulose was dissolved in IPA:water (90:10 ratio) under continuous stirring. Granules were dried. Ethyl cellulose coated granules were blended with Maltodextrin, Mannitol 160 C, Aspartame, Aerosil, Xylitol and Orange flavour in a blender for 15 minutes. Obtained granules were packaged into appropriate containers/sachets or compressed into tablets.

Stability testing: The Vitamin-C mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 9 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |
| Assay (%) | 100.6 | 101.2 | 100.8 |
| Water contents | 4.8% | 5.1% | 5.0% |

### Example 10: Encapsulation Using Polymer: Particulate Coating

| S. No | Ingredients | Quantity (% w/w) |
|---|---|---|
| 1. | Loperamide hydrochloride | 2.000 |
| 2. | Lactose | 28.000 |
| 3. | Ethyl cellulose | 5.000 |
| 4. | Mannitol SD 100 | 31.000 |
| 5. | Aspartame | 3.000 |
| 6. | Xylisorb 300 | 30.000 |
| 7. | Pine apple flavour | 1.000 |
| 8. | Purified water | QS |
| 9. | Isopropyl alcohol | QS |

Manufacturing Process:

Loperamide hydrochloride is dissolved in sufficient amount of isopropyl alcohol under continuous stirring. Lactose were loaded into fluid bed processor, sprayed loperamide hydrochloride solution and coated using ethyl cellulose solution to attain fine coated granules. Ethyl cellulose was dissolved in IPA:water (90:10 ratio) under continuous stirring. Granules were dried. Ethyl cellulose coated granules were blended with Mannitol SD 100, Aspartame, Xylisorb 300, Pine apple flavour in a blender for 15 minutes. Obtained granules were packaged into appropriate containers/sachets or compressed into tablets.

Stability testing: The Loperamide hydrochloride mouth dissolving compositions manufactured and stability studied for 6-month at the conditions 25° C./60% relative humidity and 40° C./75% relative humidity, respectively.

Stability Results:

Pack: Alu- Alu pouch

| | Stability data of Example - 10 | | |
|---|---|---|---|
| | | 40° C./75% | 25° C./60% |
| | Initial | (6 months) | (6 months) |
| | Description | | |
| | White | White | White |
| | granular | granular | granular |
| Parameters | powder | powder | powder |

Related Substances

| | | | |
|---|---|---|---|
| Impurity - F | 0.12 | 0.18 | 0.16 |
| Maximum individual | 0.08 | 0.09 | 0.09 |
| other impurity | | | |
| Total impurity | 0.10 | 0.09 | 0.09 |
| (Excluding Impurity - F) | | | |
| Assay (%) | 100.3 | 101.0 | 99.6 |
| Water contents | 3.3% | 4.1% | 4.0% |

We claim:

1. A novel process for the preparation of rapid melt granules using particulate coating comprising the steps of encapsulation of active ingredient with encapsulation polymer and/or pore former, drying, blending with one or more excipients selected from diluents, glidants, salivating agent, flavours, natural or artificial sweeteners.

2. The process for the preparation of rapid melt granules as claimed in claim 1, wherein said process comprising the steps of:
(a) coating of active ingredient using low viscous swellable or non swellable encapsulating polymers and/or pore former and diluent in a suitable equipment,
(b) drying and sifting of the coated granules obtained in step (a),
(c) blending of coated granules with excipients like additional diluents, glidants, salivating agent, flavours, natural or artificial sweeteners, and
(d) filling the obtained blend of granules into sachets.

3. The process for the preparation of rapid melt granules as claimed in claim 2, wherein said process comprising steps of:
(a) coating of active ingredient using ethyl cellulose using suitable solvent in a fluid bed processor,
(b) drying and sifting of the coated granules obtained in step (a),
(c) blending of coated granules with excipients such as maltodextrose, mannitol, xylitol, citric acid monohydrate, Aspartame and lime flavour,
(d) filling the obtained blend of granules into sachets or compressing into tablets.

4. The process for the preparation of rapid melt granules as claimed in claim 1, wherein said encapsulation polymer or pore former is selected from hydroxypropyl methylcellulose, ethyl cellulose, PEG, Eudragits, Polyvinyl alcohols, Polyvinyl acetate, hydroxypropylcellulose, hydroxypropyl-methylcellulose phthalate, cellulose acetate phthalate, starch etc, wherein concentration of encapsulation polymer or pore former is 0.1% to 30% (w/w) of the total weight of the composition.

5. The process for the preparation of rapid melt granules as claimed in claim 1, wherein said diluents are selected from silicon dioxide, sugars, starches, lactose, sucrose, sorbitol, fructose, talc, stearic acid, magnesium aluminium metasilicate, dicalcium phosphate, erythitol, xylitol, mannitol, maltitol, isomalt, dextrose, maltose, lactose, microcrystalline celluloses and mixtures thereof, wherein concentration of diluents is 1% to 95% (w/w) of the total weight of the composition.

6. The process for the preparation of rapid melt granules as claimed in claim 1, wherein said glidants are selected from talc, magnesium silicate, colloidal silicon dioxide, amorphous silicon dioxide and calcium silicate, wherein concentration of glidants is 0.1% to 3% (w/w) of the total weight of the composition.

7. The process for the preparation of rapid melt granules as claimed in claim 1, wherein said salivating agent is selected from citric acid, malic acid, tartaric acid, food salts such as sodium chloride and salt substitutes, potassium chloride, and mixtures thereof, wherein concentration of salivating agent is 0.1% to 5% (w/w) of the total weight of the composition.

8. The process for the preparation of rapid melt granules as claimed in claim 1, wherein said flavour agent is selected from volatile oils, synthetic flavor oils, flavoring aromatics, oils, liquids, oleoresins or extracts derived from plants, leaves, flowers, fruits, stems and combinations thereof, wherein concentration of flavour agent is 0.01% to 2% (w/w) of the total weight of the composition.

9. The process for the preparation of rapid melt granules as claimed in claim 1, wherein sweetener is selected from fructose (corn syrup), dextrose, invert sugar, fructose, and mixtures thereof; Acesulfame potassium, saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, xylitol, and the like, wherein concentration of sweetener is 0.1% to 5% (w/w) of the total weight of the composition.

10. A process for preparing Rapid melt granules using particulate coating, wherein said process comprising steps of:
(a) coating of active ingredient using low viscous swellable or non swellable polymers and/or pore former and optionally other pharmaceutically acceptable excipients in a suitable equipment,
(b) drying and sifting of the coated granules obtained in step (a),
(c) coating of dried granules if required using a polymer solution containing plasticizers, anti-tacking agents and optionally buffering agents, flavours and natural or artificial sweeteners,
(d) optionally drying and sifting of the coated granules obtained in step (b) or step (c),
(e) blending of coated granules with excipients such as diluents, glidants, flavours, natural or artificial sweeteners, buffering agents etc, and
(f) filling the obtained blend of granules into sachets or compressing into tablets.

11. The process for the preparation of rapid melt granules as claimed in claim 10, wherein said process comprising steps of:
(a) dissolving hydroxypropyl methylcellulose in purified water,
(b) coating of active ingredient and Mannitol mixture using hydroxypropyl methylcellulose solution,
(c) coating the hydroxypropyl methylcellulose coated granules with ethyl cellulose, Isopropyl alcohol and water solution in a fluid bed processor,
(d) drying and sifting of the coated granules obtained in step (c),
(e) blending of coated granules with talc, maltodextrin, xylitol, aspartame and lime flavour,
(f) filling the obtained blend of granules into sachets or compressing into tablets.

12. A process for preparing Rapid melt granules, wherein said process comprising steps of:
(a) complexing of active ingredient with sodium polystyrene sulfonate,
(b) draining the supernatant solution and collecting the residue,
(c) drying and sifting of complexed granules,
(d) blending of complexed granules with talc, Aspartame, Sorbitol, Peach flavour, and Maltodextrin, and
(e) filling the obtained blend of complexed granules into sachets or compressing into tablets.

13. The process for the preparation of rapid melt granules as claimed in claim 12, wherein said process comprising steps of:
(a) complexing of active ingredient with sulfonated polymers of ion exchange resin,
(b) draining the supernatant solution and collecting the residue,
(c) drying and sifting of complexed granules,
(d) coating the complex with using low viscous swellable or non swellable polymers and/or pore former,
(e) blending of complexed granules with excipients such as diluents, glidants, flavours, natural or artificial sweeteners, buffering agents etc, and
(f) filling the obtained blend of complexed granules into sachets or compressing into tablets.

14. The process for the preparation of rapid melt granules as claimed in claim 12, wherein said process comprising steps of:
(a) complexing of active ingredient with sodium polystyrene sulfonate,
(b) draining the supernatant solution and collecting the residue,
(c) drying and sifting of complexed granules,
(d) coating the complexed granules with ethyl cellulose with suitable solvent,
(e) blending of coated complexed granules with aspartame, xylitol, orange flavour, mannitol and talc, and
(f) filling the obtained blend of complexed granules into sachets or compressing into tablets.
